(19) United States
(12) Patent Application Publication
(10) Pub. No.: US 2018/0303675 A1
(43) Pub. Date: Oct. 25, 2018

(54) FACTOR CONCENTRATION GAUZE COMBINED WITH ENDOTHERMIC REACTION USED TO COMBAT VERY PROXIMAL TRAUMATIC AMPUTATION HEMORRHAGING

(71) Applicants: Hannah Mae Jones, San Antonio, TX (US); Madeleine Marie Farrer, San Antonio, TX (US); Brian Nathaniel Ke-Han Ruliffson, San Antonio, TX (US); Jose Hernandez Trevino, Uvalde, TX (US); Christopher Rathbone, San Antonio, TX (US)

(72) Inventors: Hannah Mae Jones, San Antonio, TX (US); Madeleine Marie Farrer, San Antonio, TX (US); Brian Nathaniel Ke-Han Ruliffson, San Antonio, TX (US); Jose Hernandez Trevino, Uvalde, TX (US); Christopher Rathbone, San Antonio, TX (US)

(21) Appl. No.: 15/953,288

(22) Filed: Apr. 13, 2018

Related U.S. Application Data
(60) Provisional application No. 62/489,451, filed on Apr. 24, 2017.

(51) Int. Cl. A61F 13/00; A61L 15/60; A61L 15/42; A61L 15/44; A61L 15/26; A61L 15/24

(52) U.S. Cl. CPC A61F 13/00063; A61F 13/00029; A61L 15/60; A61L 15/42; A61L 2400/04; A61L 15/26; A61L 15/24; A61F 2013/00106; A61F 2013/00153; A61L 15/44

(57) ABSTRACT

The disclosed invention is a device and method of use for gauze utilized in the treatment of hemorrhage. In an embodiment the device combines clotting factor concentrator gauze and urea in order to slow hemorrhage and prevent hemorrhage induced coagulopathy. In an embodiment, the gauze is comprised of a first layer comprised of gauze and a second layer embedded with urea. The urea reacts spontaneously when it comes into contact with water, and undergoes an endothermic reaction. This endothermic reaction cools the smaller blood vessels exposed from soft tissue trauma, causing enhanced platelet activity as well as vasoconstriction to minimize the bleeding that occurs before and after the clotting cascade is activated over the entire wound surface.

### CROSS-REFERENCE TO RELATED APPLICATIONS

This application claims the benefit under Title 35 United States Code § 119(e) of U.S. Provisional Patent Application Ser. No. 62/489,451; Filed: Apr. 24, 2017, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

This device relates to a field of the detection of medical devices to slow or stop bleeding.

### BACKGROUND OF THE INVENTION

Hemorrhage resulting from a traumatic injury is often the major cause of possible complications and death. Hemostatic agents may provide one of the easiest and most effective methods of treating hemorrhage. The disclosed invention provides for improvements to the treatment of hemorrhage.

### BRIEF SUMMARY OF THE INVENTION

Certain embodiments are directed to a device that combines clotting factor concentrator gauze and urea in order to slow hemorrhage and prevent hemorrhage induced coagulopathy.

In some embodiments a hemorrhage treatment device is comprised of a first (outer) layer that is placed into contact with the patient, said layer having the property of absorbing the water content of the blood, thereby leaving all of the clotting factors on the surface to build up and catalyze the clotting cascade much earlier than what would occur naturally. In some embodiments the first layer is a clotting factor concentrating gauze incorporating kaolin such as Quickclot Combat Gauze.

In some embodiments the first layer is a gauze treated with any combination of hemostatic agents including, but not limited to include zeolite, smectite, kaolin, chitosan, and plant-derived polysaccharides.

In some embodiments of the invention, adjacent to the first layer is a second layer wherein solid chemical-grade urea granules are embedded. The urea reacts spontaneously when it comes into contact with water, which will be supplied from the first layer, and undergoes an endothermic reaction. This endothermic reaction cools the smaller blood vessels exposed from soft tissue trauma, causing enhanced platelet activity as well as vasoconstriction to minimize the bleeding that occurs before and after the clotting cascade is activated over the entire wound surface.

In some embodiments the second layer containing urea is surrounded by a material selected to block ultra-violet radiation while also being mechanically strong. In some embodiments this material may be 4 mil black polyethylene film. In some embodiments sodium polyacrylate, or some other superabsorbent polymer (SAP) is mixed into the urea granule layer to minimize any dissolved urea from entering the wound site and to additionally insulate the urea chamber. The SAP may swell when it comes into contact with the water component of blood and forms a hydrogel matrix that may encase the urea granules. The SAP increases the absorbance capacity of the device.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a cross-sectional side view of the factor concentrator gauze in accordance with embodiments of the disclosure;

FIG. 2 is an exploded perspective view of the factor concentrator gauze in accordance with embodiments of the disclosure;

FIG. 3 is an embodiment of the factor concentrator gauze utilizing a 4 mil black polyethylene plastic film in accordance with embodiments of the disclosure;

FIG. 4 is an embodiment of the factor concentrator gauze utilizing rayon polyester in accordance with embodiments of the disclosure;

FIG. 5 is an embodiment of the factor concentrator gauze utilizing silnylon ripstop 20D in accordance with embodiments of the disclosure;

FIG. 6 is an embodiment of the factor concentrator gauze utilizing urea and a sodium polyacrylate layer in accordance with embodiments of the disclosure;

FIG. 7 is a perspective view of an embodiment of the factor concentrator gauze utilizing a 4 mil black polyethylene plastic film in accordance with embodiments of the disclosure;

FIG. 8 is a perspective view of the factor concentrator gauze utilizing silnylon ripstop 20D in accordance with embodiments of the disclosure.

FIG. 9 is a perspective view of the factor concentrator gauze utilizing urea and a sodium polyacrylate layer in accordance with embodiments of the disclosure;

FIG. 10 is a perspective view of the factor concentrator gauze utilizing rayon polyester in accordance with embodiments of the disclosure.

### DETAILED DESCRIPTION OF THE INVENTION

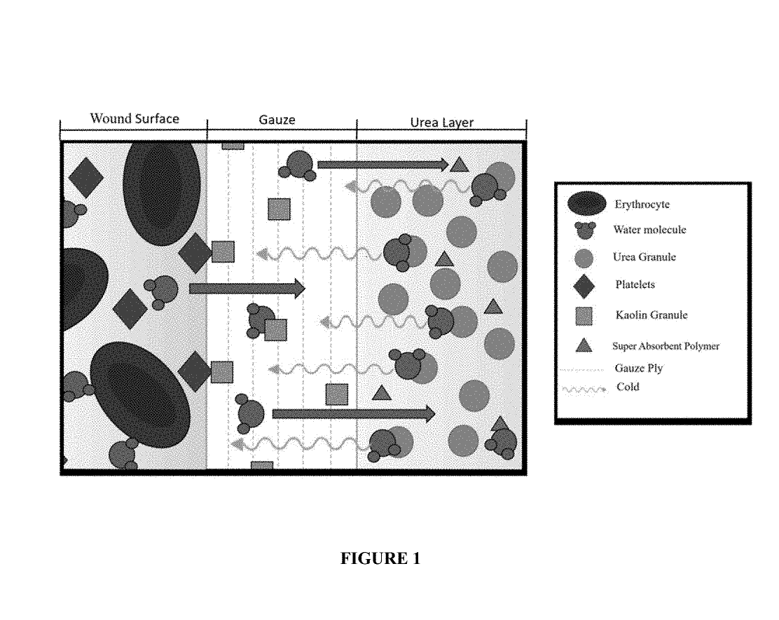

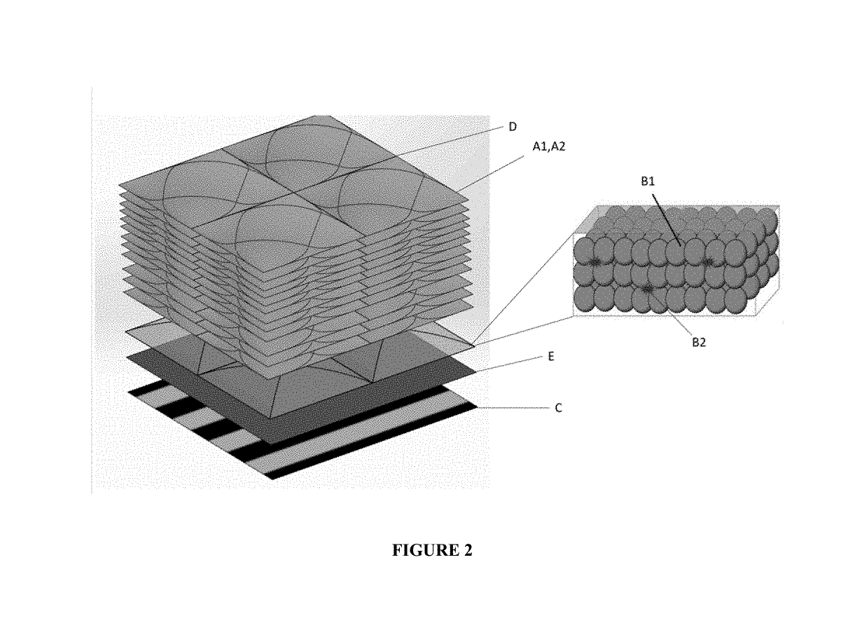

FIG. 1 illustrates an embodiment of the hemmorage control device. FIG. 2 illustrates the multiple layers of an embodiment of a hemorrhage control device. The various layers shown are A1. Rayon Polyester Gauze, A2. Kaolin Granules, B1. Solid Urea Granules, B2. Sodium Polyacrylate, C. 4 mil Black Polyethylene Film, D. Gutermann Polyester Thread, E. Silnylon Fabric, F. Loctite Vinyl, Fabric & Plastic Flexible Adhesive, G. X-Ray Detectable Thread.

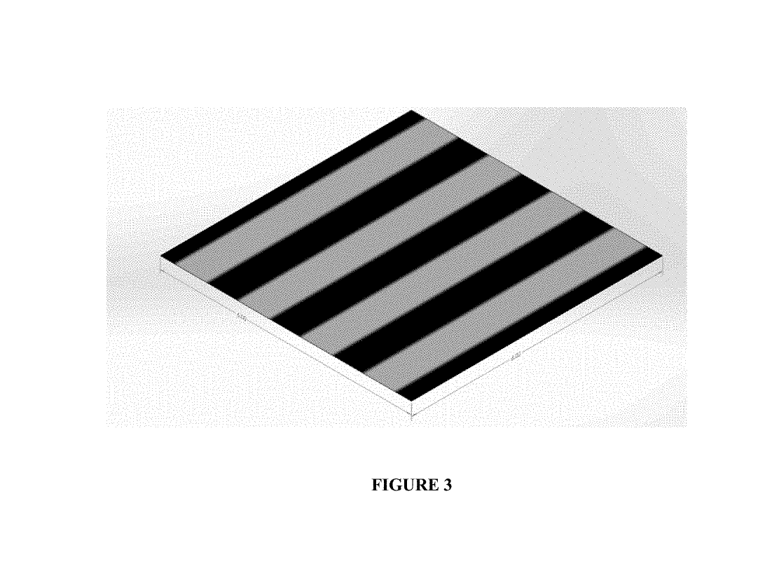

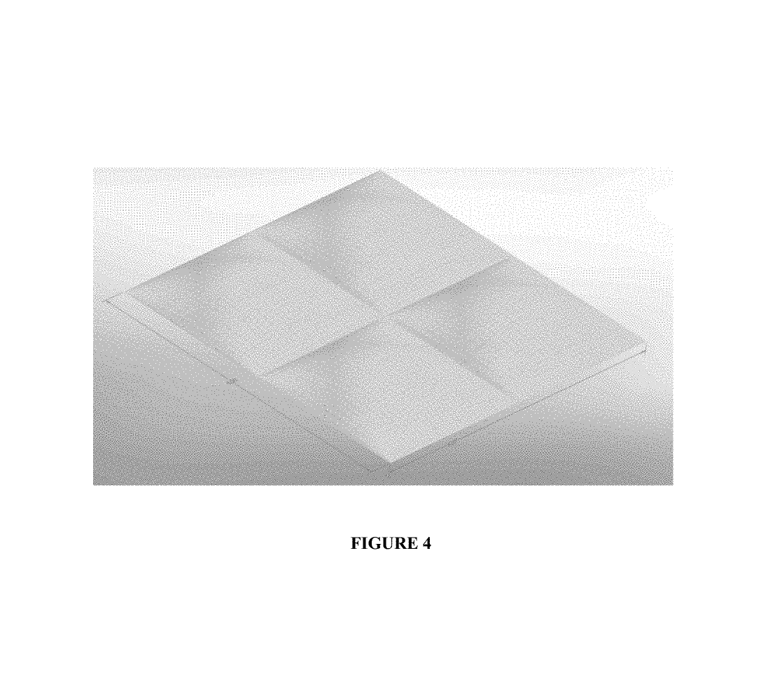

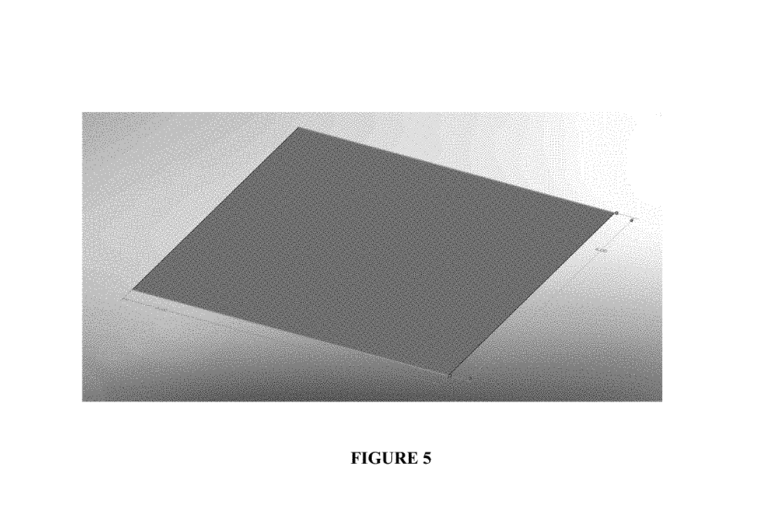

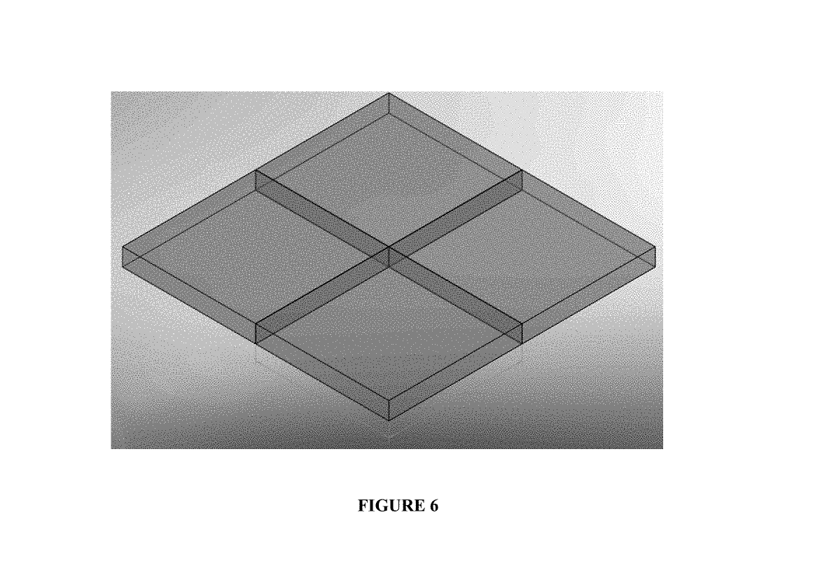

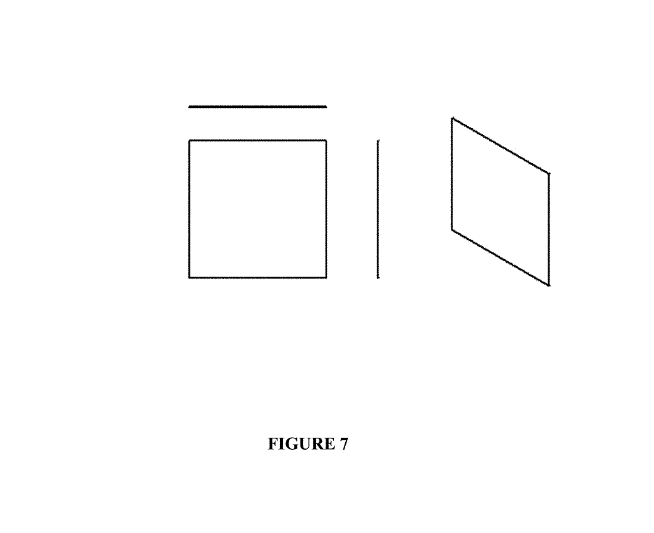

A complete hemorrhage control device may be assembled by using white marine enamel, to paint 1″ white strips every alternating inch onto the back of the 4 mil Black Polyethylene Plastic Film as illustrated in FIG. 3. and allow to completely dry. Stack twelve layers of Rayon Polyester Gauze as illustrated in FIG. 4. and using Gutermann MARA 70 Polyester Thread, sew all four edges of the gauze together. Then stack Silnylon Ripstop 20D as illustrated in FIG. 5. below of the assembled gauze and using Gutermann MARA 70 Polyester Thread, sew three edges of the silnylon and gauze bandage structure together and using Gutermann MARA 70 Polyester Thread, sew three edges of the silnylon and gauze bandage structure together. Then, using Gutermann MARA 70 Polyester Thread, sew a vertical center line, 3″ from the side, through the silnylon and gauze bandage structure. As illustrated in FIG. 6, grind 200 g of urea granules into a fine powder and aliquot into four equal mass groups, add 0.25 g of Sodium Polyacrylate to each aliquot and thoroughly mix. Then pour one aliquot into the bottom left pocket located between the silnylon and gauze Then repeat that for the bottom right pocket. Then you will sew a horizontal line 3″ above the base in order to seal the pockets created in the previous steps. You will repeat these steps for the top left and top right pockets. Generously coat the unpainted side of the 6″×6″ 4 mil Black Polyethylene Plastic Film as illustrated in FIG. 7. and the exposed silnylon with Loctite Vinyl, Fabric & Plastic Flexible Adhesive. Finally place one coated surface onto the other and submit to compression for 24 hours to allow the loctite adhesive to cure.

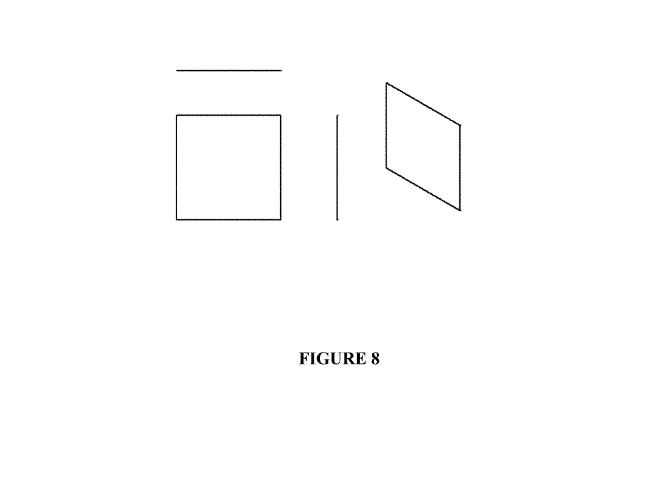

FIG. 8 is a perspective view of the factor concentrator gauze utilizing silnylon ripstop 20D in accordance with embodiments of the disclosure.

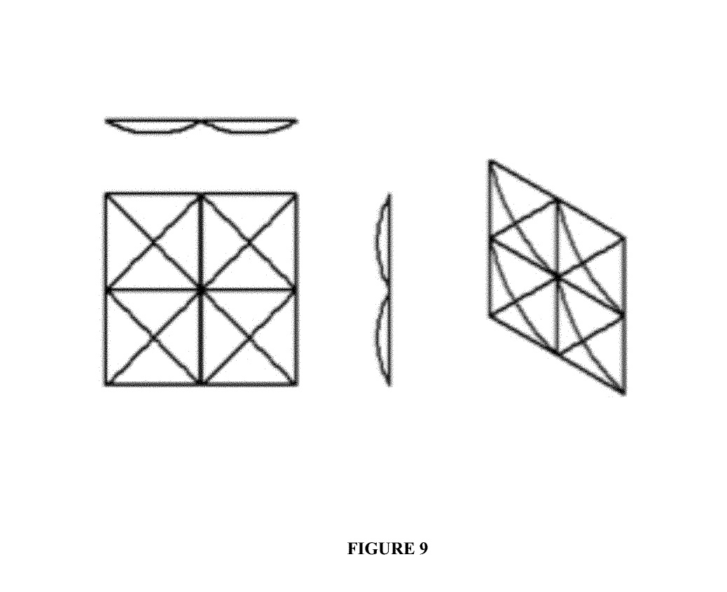

FIG. 9 is a perspective view of the factor concentrator gauze utilizing urea and a sodium polyacrylate layer in accordance with embodiments of the disclosure.

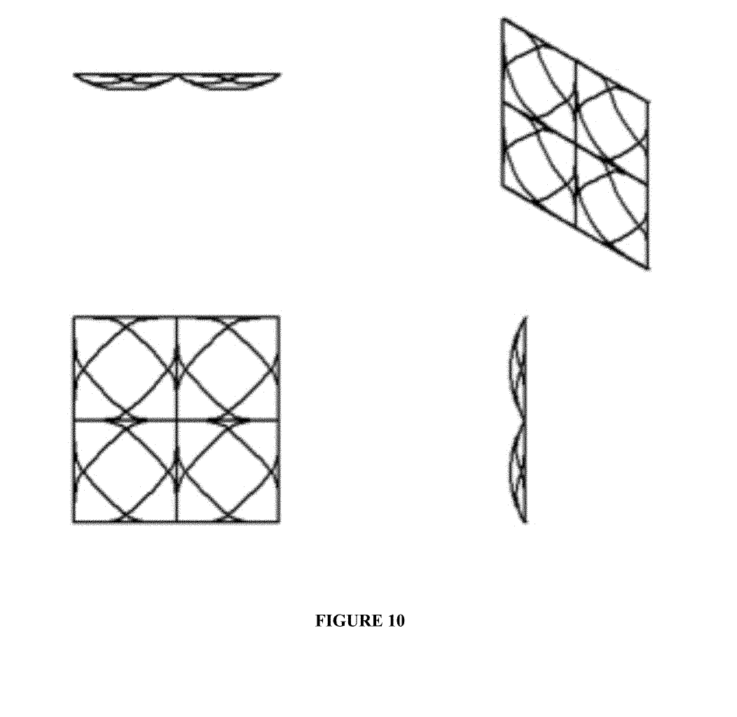

FIG. 10 is a perspective view of the factor concentrator gauze utilizing rayon polyester in accordance with embodiments of the disclosure.

In brief, as described herein provides for gauze utilized in the treatment of hemorrhage.

The disclosed devices and methods are generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention.

Terms such as “a”, “an”, and “the” are not intended to refer to only a singular entity, but include the general class of which a specific example may be used for illustration. The terminology herein is used to describe specific embodiments of the invention, but their usage does not delimit the disclosed devices or methods, except as may be outlined in the claims.

Any embodiments comprising a one component or a multi-component system having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific devices and methods described herein. Such equivalents are considered to be within the scope of this invention and are covered by the claims.

All publications, references, patents, and patent applications mentioned in the specification are indicative of the level of those skilled in the art to which this invention pertains. All publications, references, patents, and patent application are herein incorporated by reference to the same extent as if each individual publication, reference, patent, or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The devices and/or methods disclosed and claimed herein can be made and executed without undue experimentation in light of the present disclosure. While the devices and methods of this invention have been described in terms of preferred embodiments, it will be apparent to those skilled in the art that variations may be applied to the devices and/or methods and in the steps or in the sequence of steps of the method described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. What is claimed is a device for controlling hemorrhage comprising:
a first layer that is placed into contact with the patient's wound, said layer having the property of absorbing the water content of the blood;
a second layer containing urea, said layer configured so that the urea reacts when it comes into contact with water supplied from the first layer, and undergoes an endothermic reaction;
and wherein said second layer has a surface in contact with a surface of said first layer.

2. The device of claim 1, wherein said first layer is gauze.

3. The device of claim 1, wherein said first layer is also comprised of a hemostatic agent.

4. The device of claim 3, wherein said hemostatic agent is selected from the group zeolite, smectite, kaolin, plant-based polysaccharides, and chitosan.

5. The device of claim 1, wherein said urea is solid chemical grade granules embedded in said second layer.

6. The device of claim 1, wherein said urea is 99.9 percent pure urea embedded in said second layer.

7. The device of claim 1, wherein said urea is 99.9 percent pure urea in powder form embedded in said second layer.

8. The device of claim 1, wherein said second layer is surrounded by a material to block ultraviolet radiation.

9. The device of claim 8, wherein said material is 4 mil black polyethylene.

10. The device of claim 1, wherein said second layer is further comprised of sodium polyacrylate mixed with said urea.

11. The device of claim 1, wherein said second layer is further comprised of super absorbent polymer (SAP) mixed with said urea.

12. The device of claim 2, wherein said wherein said first layer is also comprised of a hemostatic agent.

13. The device of claim 12, wherein said urea is solid chemical grade granules embedded in said second layer.

14. The device of claim 13, wherein said second layer is surrounded by a material to block ultraviolet radiation.

15. The device of claim 14, wherein said second layer is further comprised of sodium polyacrylate mixed with said urea.

16. The device of claim 14, wherein said second layer is further comprised of super absorbent polymer (SAP) mixed with said urea.

17. The device of claim 14, wherein said second layer outer surface is comprised of white marine stripes.
